(19) United States
(12) Patent Application Publication
(10) Pub. No.: US 2007/0142433 A1
(43) Pub. Date: Jun. 21, 2007

(54) Angiogenesis-affecting compounds and methods of use thereof

(76) Inventors: Helmut Augustin, Freiburg (DE); Mats Hellstrom, Uppsala (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/582,570

(22) Filed: Oct. 17, 2006

Related U.S. Application Data
(63) Continuation-in-part of application No. 11/541,861, filed on Oct. 2, 2006.
Application No. 11/541,861 is a continuation of application No. PCT/SE05/00506, filed on Apr. 6, 2005.
(60) Provisional application No. 60/521,346, filed on Apr. 6, 2004.
(60) Provisional application No. 60/596,737, filed on Oct. 18, 2005.

(51) Int. Cl. A61K 31/445

(52) U.S. Cl. 514/317

(57) ABSTRACT

Angiogenesis may be affected by administering a compound to a group of cells, a tissue or an organism. Such affect may be to inhibit or stimulate angiogenesis. The compounds may be used to treat disease states related to insufficient or unregulated angiogenesis.

### CROSS-REFERENCE TO RELATED APPLICATIONS

This application is a Continuation-in-Part Application of U.S. Ser. No. 11/541,861 filed Oct. 2, 2006, which is a U.S. National Stage application of International Application Serial No. PCT/SE2005/000506 filed on Apr. 6, 2005, which claims the benefit of U.S. Provisional Application Ser. No. 60/521,346 filed Apr. 6, 2004. This application also claims the benefit of U.S. Provisional Application Ser. No. 60/596,737 filed Oct. 18, 2005.

### BACKGROUND OF THE INVENTION

1. Field of the Invention

The invention relates to compounds useful for promoting or inhibiting angiogenesis and to pharmaceutical compositions containing a compound that affects angiogenesis, which are useful in a number of angiogenesis-related conditions. Further, the invention relates to methods of using such angiogenesis-affecting compounds.

2. Description of the Related Art

Angiogenesis is the fundamental process by which new blood vessels are formed. The process involves the migration of vascular endothelial cells into tissue, followed by the condensation of such endothelial cells into vessels. Angiogenesis may be induced by an angiogenic agent or be the result of a natural condition. Angiogenesis, or its absence, plays an important role in the maintenance of a variety of pathological states. Some of these states are characterized by neovascularization, e.g., cancer, diabetic retinopathy, glaucoma, and age related macular degeneration. Others, e.g., stroke, infertility, heart disease, ulcers, and scleroderma, are diseases of angiogenic insufficiency.

Angiogenesis has a number of stages (see, e.g., Zhu and Witte, Invest New Drugs 17:195-212, 1999). The early stages of angiogenesis include endothelial cell protease production, migration of cells, and proliferation. The early stages also appear to require some growth factors. Later stages of angiogenesis include population of the vessels with mural cells, basement membrane production, and the induction of vessel bed specializations. The final stages of vessel formation include remodelling, wherein a forming vasculature becomes a stable, mature vessel bed. Thus, the process is highly dynamic, often requiring coordinated spatial and temporal waves of gene expression.

The complex angiogenesis process is subject to disruption through interference with one or more critical steps, and numerous disease states can result from or be exacerbated by the disruption. Unregulated angiogenesis can cause or worsen disease, for example, ocular neovascularization has been implicated as the most common cause of blindness and underlies the pathology of approximately 20 eye diseases. In certain previously existing conditions such as arthritis, newly formed capillary blood vessels invade the joints and destroy cartilage. In diabetes, new capillaries formed in the retina invade the vitreous humour, causing bleeding and blindness.

In addition to pathologies linked to unregulated angiogenesis, insufficient angiogenesis can also lead to undesirable results. Dead or damaged tissue can lead to numerous pathologies, revascularization of damaged tissues through a healthy, normal angiogenic process is essential to preventing further complications.

Thus, it has been recognized in the medical art that compounds which affect angiogenesis have potential both as treatments for a number of disease states and as research tools. Investigators have made some progress in identifying and utilizing natural angiogenesis-affecting molecules. Some progress has also been made toward identifying compounds which could be used to affect angiogenesis. Often it is revealed that those compounds which have promising effects on angiogenesis have unacceptable toxicity profiles, are prohibitively difficult or expensive to make, or both. Despite the current level of knowledge and available treatments, there remains a need in the art for further medicaments or compounds that can be utilized for affecting angiogenesis or preventing or treating an angiogenesis-related condition.

Perhexiline was initially used for management of angina pectoris. Despite some positive effects, adverse hepatic and neurological effects were also associated with Perhexiline administration. The original classification as a coronary vasodilator was revised to a calcium channel antagonist, but recent data suggest it may be a cardiac metabolic agent acting through the inhibition of the enzyme carnitine palmitoyltransferase-1 (CPT-1). Killalea S. M. and Krum H. describe an evaluation of Perhexiline for managing severe myocardial ischemia in Am. J. Cardiocasc. Drugs, 2001; 1(3):193-204.

Erythromycin is produced by a strain of Streptomyces erythraeus belonging to the macrolide group of antibiotics. It is basic and readily forms salts with acids, however, it is the base which is microbiologically active. It is primarily given topically or orally to treat bacterial infections. Given the substantial history for use of this drug in humans, it would be desirable to elucidate any further applications for which it would be a candidate. Some recent research has indicated that erythromycin may have new applications, see, for example, Yatsunami J, et al., Inhibitory effects of roxithromycin on tumor angiogenesis, growth and metastasis of mouse B16 melanoma cells, Clin Exp Metastasis. 1999 March;17(2):119-24; Yatsunami J, et al., Inhibition of tumor angiogenesis by roxithromycin, a 14-membered ring macrolide antibiotic, Cancer Lett. Sep. 25, 1998;131(2):137-43; Yatsunami J, et al., Clarithromycin is a potent inhibitor of tumor-induced angiogenesis, Res Exp Med (Berl). 1997;197(4):189-97.

Dopamine regulators are known and many have been effectively used in patients. For example, Bromocriptine mesylate (Bromocriptine) is an ergot derivative with potent dopamine receptor antagonist activity. It can be used in various other applications, such as infertility, but its full range of potential uses has not yet been elucidated. Similarly, Eticlopride and Lisuride are described in the relevant literature for their ability to regulate dopamine. Dopamine and Dopamine receptor-2 selective agonists such as Bromocriptine have been shown to prevent angiogenesis in Basu S et al., The neurotransmitter dopamine inhibits angiogenesis induced by vascular permeability factor/vascular endothelial growth factor, Nat Med. 2001 May;7(5):569-74. Eticlopride was shown to have not effect on angiogenesis.

It is also known in the art that anti-mycotics exist. For example, miconazole, sulconazole and econazole are known substances that have been utilized in various treatment settings for their anti-fungal activity. Miconazole is perhaps the best known, but sulconazole is also an anti-mycotic, an imidazole derivative with known broad-spectrum antifungal activity. Its ability to act against other microbes is being explored. Econazole, like other anti-fungals, is typically administered topically. When this is done very low doses of the active substance are absorbed. This can be a detriment both in elucidating other uses and in utilizing econazole for those prospective other uses.

Further, anti-androgens have been provided, examples of which include flutamide and danazol. Flutamide has been successfully used to inhibit androgen, however, there is a serious and substantial risk of hepatic injury in conjunction with its use. Liver failure and death have been reported, typically within the initial few months of flutamide use. Despite this, determining any other potentially beneficial uses of flutamide is warranted. The synthetic hormone danazol, derived from ethisterone, could be a desirable medicament due to its rapid and extensive metabolization following oral administration. Unlike flutamide which is primarily used in male patients, danazol also offers the advantage of having clinical data in both male and female subjects.

### SUMMARY OF THE INVENTION

Therefore, it is an object of the present invention to provide compounds that affect angiogenesis. It is a further object of the invention to provide methods of using angiogenesis-affecting compounds. Yet another object of the invention is to elucidate new uses for known compounds.

According to a first embodiment, a method of inhibiting angiogenesis in a group of cells, a tissue or an organism is provided which comprises administering a therapeutically effective amount of perhexiline or a combination of erythromycin and at least one anti-VEGF treatment or pharmaceutically acceptable prodrugs, salts, solvates, hydrates, active metabolites, or combinations thereof to the group of cells, tissue or organism. The organism can be a mammal, and the mammal can have a condition selected from the group consisting of cancer, sarcomas, retinopathy, macular degeneration, corneal ulceration, scleroderma, Berger's disease, proliferative vitreoretinopathy, chronic inflammation, inflammatory bowel disease, psoriasis, sarcoidosis, and rheumatoid arthritis. The compound can be administered locally.

According to a further embodiment, a method of inhibiting angiogenesis in a group of cells, a tissue or an organism is provided which comprises administering a therapeutically effective amount of a pharmaceutical composition comprising perhexiline or a combination of erythromycin and at least one anti-VEGF treatment or pharmaceutically acceptable prodrugs, salts, solvates, hydrates, active metabolites, or combinations thereof to the group of cells, tissue or organism. The organism can be a mammal, and the mammal can have a condition selected from the group consisting of cancer, sarcomas, retinopathy, macular degeneration, corneal ulceration, scleroderma, Berger's disease, proliferative vitreoretinopathy, chronic inflammation, inflammatory bowel disease, psoriasis, sarcoidosis, and rheumatoid arthritis. The compound can be administered locally. The pharmaceutical composition can be formulated as a salve, gel, ointment, patch, injection, oral solution or suspension and can be in a controlled release matrix. The pharmaceutical composition can further comprise a pharmaceutically acceptable carrier, diluent, vehicle, or excipient. Where the pharmaceutical composition comprises perhexiine the method can further comprise administering at least one anti-VEGF treatment to the group of cells, tissue, or organism. An anti-VEGF treatment can be AVASTIN or MACUGEN.

According to a further embodiment, a method of stimulating angiogenesis in a group of cells, a tissue or an organism is provided which comprises administering a therapeutically effective amount of a compound selected from the group consisting of Bromocriptine, Eticlopride, Lisuride (S)(−), Miconazole, Sulconazole, Econazole, Flutamide and Danazol or pharmaceutically acceptable prodrugs, salts, solvates, hydrates, active metabolites, or combinations thereof to the group of cells, tissue or organism. The organism can be a mammal. The mammal can have a condition selected from the group consisting of stroke, ischemic heart disease, wound healing, ischemia, myocardial infarction, myocardosis, angina pectoris, unstable angina, coronary arteriosclerosis, arteriosclerosis obliterans, and cerebral infarction. The compound may be administered locally.

According to a further embodiment, a method of stimulating angiogenesis in a group of cells, a tissue or an organism is provided which comprises administering a therapeutically effective amount of a pharmaceutical composition comprising a compound selected from the group consisting of Bromocriptine, Eticlopride, Lisuride (S)(−), Miconazole, Sulconazole, Econazole, Flutamide and Danazol or pharmaceutically acceptable prodrugs, salts, solvates, hydrates, active metabolites, or combinations thereof to the group of cells, tissue or organism. The organism can be a mammal. The mammal can have a condition selected from the group consisting of stroke, ischemic heart disease, wound healing, ischemia, myocardial infarction, myocardosis, angina pectoris, unstable angina, coronary arteriosclerosis, arteriosclerosis obliterans, and cerebral infarction.

According to a further embodiment, a method of increasing blood flow to a tissue is provided which comprises administering a pharmaceutical composition comprising a compound selected from the group consisting of Bromocriptine, Eticlopride, Lisuride (S)(−), Miconazole, Sulconazole, Econazole, Flutamide and Danazol to the tissue in an amount effective to promote angiogenesis.

For convenience, certain terms employed in the specification, examples, and appended claims are collected here.

The term “patient” refers to the subject of the novel treatment disclosed herein.

The term “tissue” refers to a group of living cells. The term should be read to encompass single cells and groups of cells where appropriate. The term can also be read to include organs. The term refers not only to living material in situ but also to living material that has been cultured in vitro or extracted from an organism.

The term “animal” or “organism” refers to mammals, preferably humans. The term “angiogenesis” means the process by which living cells, tissues, or organisms form new blood vessels.

The term “angiogenesis-affecting” means a compound or process which influences angiogenesis in a treated tissue or organism. The effect could be, for example, inhibition or promotion of angiogenesis.

The phrase “angiogenesis-related condition” refers to any one of the medical conditions or disease states recognized to be influenced by angiogenesis or by an increase/decrease in angiogenesis of by the lack thereof, including conditions which may be linked to angiogenesis in the future. Examples of such conditions include cancer, sarcoma, retinopathy, macular degeneration, corneal ulceration, stroke, ischemic heart disease, infertility, ulcers, scleradoma, wound healing, ischemia, ischemic heart disease, myocardial infarction, myocardosis, angina pectoris, unstable angina, coronary arteriosclerosis, arteriosclerosis obliterans, Berger's disease, arterial embolism, arterial thrombosis, cerebrovascular occlusion, cerebral infarction, cerebral thrombosis, cerebral embolism, rubeosis proliferative vitreoretinopathy, chronic inflammation, inflammatory bowel disease, psoriasis, sarcoidosis, and rheumatoid arthritis.

The term “stimulant” refers to molecules or compounds which initiate, promote, or increase in speed, duration, or degree the natural or typical (i.e., unaffected) angiogenesis in the treated tissue or patient.

The term “inhibitor” refers to molecules or compounds which stop, prevent, reduce, hinder, or delay the natural or typical (i.e., unaffected) angiogenesis in the treated tissue or patient.

The term “pharmaceutical,” “pharmaceutical compound,” or “drug” as used herein, refers to any medicinal substance used in living cells, tissues, humans or other animals. Encompassed within this definition are compound analogs, naturally occurring and synthetic compounds.

The phrase “pharmaceutically acceptable” refers to molecular entities and compositions that are physiologically tolerable and do not typically produce an allergic or similar untoward reaction when administered to a patient.

The phrase “therapeutically effective amount” is used herein to mean an amount which, when applied as part of a desired dosage regimen, is sufficient to affect angiogenesis to an extent that results in some clinically significant change. When used in conjunction with angiogenesis inhibitors, the term means an amount which will prevent, or preferably reduce by at least about 30 percent, more preferably by at least about 50 percent, most preferably by at least about 90 percent, angiogenesis in the tissue or patient treated. When used in conjunction with angiogenesis stimulators or promoters, the term means an amount which will start, or preferably increase by at least about 30 percent, more preferably by at least about 50 percent, most preferably by at least about 90 percent, angiogenesis in the tissue or patient treated. The amount of a given compound described herein that will correspond to a “therapeutically effective amount” will vary depending upon factors such as the particular compound, the disease condition and the severity thereof, the identity of the mammal in need thereof, but it can nevertheless be readily determined by one of skill in the art.

“Treating” or “treatment” is intended to mean at least the mitigation of a disease condition in a mammal, such as a human, that is alleviated by affecting angiogenesis, and includes: (a) prophylactic treatment in a mammal, particularly when the mammal is found to be predisposed to having the disease condition but not yet diagnosed as having it; (b) inhibiting the disease condition; and/or (c) alleviating, in whole or in part, the disease condition.

A “pharmaceutically acceptable prodrug” means a compound that may be converted under physiological conditions or by solvolysis to a compound of one of the formulas disclosed herein.

A “pharmaceutically acceptable active metabolite” means a pharmacologically active product produced through biological metabolism, such as in the body of a patient, of a compound of one of the formulas disclosed herein.

A “pharmaceutically acceptable solvate” means a solvate that retains the biological effectiveness and properties of the biologically active compounds of one of the formulas disclosed herein. Examples of pharmaceutically acceptable solvates include, but are not limited to, one or more of the compounds disclosed herein in combination with water, isopropanol, ethanol, methanol, DMSO, ethyl acetate, acetic acid, or ethanolamine.

A “pharmaceutically acceptable salt” means a salt that retains the biological effectiveness and properties of the free acids and bases of compounds disclosed herein and that is not biologically or otherwise undesirable. Examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxyenzoates, phthalates, sulfonates, xylenesulfonates, phenylacetates, phenylpropionates, phenylbutyrates, citrates, lactates, .gamma.-hydroxybutyrates, glycollates, tartrates, methane-sulfonates, propanesulfonates, naphthalene-1-sulfonates, naphthalene-2-sulfonates, and mandelates.

If the inventive compound is a base, the desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acids such as glucuronic acid and galacturonic acid; alpha-hydroxy acids such as citric acid and tartaric acid; amino acids such as aspartic acid and glutamic acid; aromatic acids such as benzoic acid and cinnamic acid; sulfonic acids such as p-toluenesulfonic acid or ethanesulfonic acid; or the like.

If the inventive compound is an acid, the desired salt may be prepared by any suitable method known to the art, including treatment of the free acid with an inorganic or organic base, such as an amine (primary, secondary or tertiary); an alkali metal or alkaline earth metal hydroxide; or the like. Illustrative examples of suitable salts include organic salts derived from amino acids such as glycine and arginine; ammonia; primary, secondary and tertiary amines; and cyclic amines such as piperidine, morpholine, and piperazine, and inorganic salts derived from sodium, calcium, potassium, magnesium, manganese, iron, copper, zinc, aluminum, and lithium.

In the case of compounds, salts, or solvates that are solids, it is understood by those skilled in the art that the inventive compounds, salts, and solvates may exist in different crystal forms, all of which are intended to be within the scope of the present invention.

The inventive compounds may exist as single stereoisomers, racemates, and/or mixtures of enantiomers and/or diastereomers. All such single stereoisomers, racemates, and mixtures thereof are intended to be within the scope of the present invention. Preferably, the inventive compounds are used in optically pure form.

As generally understood by those skilled in the art, an optically pure compound is one that is enantiomerically pure. As used herein, the term “optically pure” is intended to mean a compound which comprises at least a sufficient amount of a single enantiomer to yield a compound having the desired pharmacological activity. Preferably, “optically pure” means a compound that comprises at least 90% of a single isomer, preferably at least 95%, more preferably 97.5%, and most preferably 99%.

The present invention is also directed to methods of affecting angiogenesis and methods of treating or preventing angiogenesis-related conditions, which methods or treatments comprise the use of a compound disclosed herein or a pharmaceutically acceptable prodrug, salt, active metabolite, or solvate thereof.

The activity of the inventive compounds as angiogenesis-effectors may be measured by any of the methods available to those skilled in the art, including in vivo and in vitro assays.

Administration of the compounds disclosed herein, or their pharmaceutically acceptable prodrugs, salts, active metabolites, and solvates, may be performed according to any of the accepted modes of administration available to those skilled in the art. Illustrative examples of suitable modes of administration include, but are not limited to, oral, nasal, parenteral, topical, and transdermal.

The compounds disclosed herein may be administered as a pharmaceutical composition in any suitable pharmaceutical form recognizable to the skilled artisan. Suitable pharmaceutical forms include, but are not limited to, solid, semisolid, liquid, or lyopholized formulations, such as tablets, powders, capsules, suspensions, and aerosols. The pharmaceutical composition may also include suitable excipients, diluents, vehicles, and carriers, as well as other pharmaceutically active agents, depending upon the intended use.

Acceptable methods of preparing suitable pharmaceutical forms of the pharmaceutical compositions are known to those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for, for example, oral, parenteral, topical, intranasal, intrabronchial and/or intraocular administration.

Solid or liquid pharmaceutically acceptable carriers, diluents, vehicles, or excipients may be employed in the pharmaceutical compositions. Illustrative solid carriers include starch, lactose, calcium sulphate dihydrate, terra alba, sucrose, talc, gelatin, pectin, acacia, magnesium stearate, and stearic acid. Illustrative liquid carriers may include alcohol, oil, saline, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g. solution), or a nonaqueous or aqueous liquid suspension.

A dose of the pharmaceutical composition contains at least a therapeutically effective amount of the active compound (i.e., a compound described herein or a pharmaceutically acceptable prodrug, salt, active metabolite, or solvate thereof) and preferably is made up of one or more pharmaceutical dosage units. The selected dose may be administered to a mammal, for example, a human patient, in need of angiogenesis-effecting treatment, by any known method of administering the dose including topical, for example, as an ointment or cream; orally; parenterally by injection; or continuously by intranasal, intrabronchial, intraaural, or intraocular infusion.

### BRIEF DESCRIPTION OF THE DRAWING FIGURES

FIG. 1 shows exemplary results of primary angiogenesis screening assays;

FIG. 2 shows the chemical structure of perhexiline maleate;

FIG. 3 shows the chemical structure of erythromycin;

FIGS. 4A, 4B, and 4C depict cell proliferation data for perhexiline;

FIGS. 5A, 5B, and 5C depict cell proliferation data for erythromycin;

FIGS. 6A, 6B, and 6C show cytoviability assay results for perhexiline;

FIGS. 7A, 7B, and 7C show cytoviability assay results for erythromycin;

FIG. 8 depicts cell viability data for B16F10 cells treated with perhexiline;

FIG. 9 depicts cell viability data for B16F10 cells treated with erythromycin;

FIG. 10 shows IC₅₀ values for two VEGFR-1 inhibitors;

FIGS. 11A and 11B show IC₅₀ values for perhexiline alone and in conjunction with VEGFR-1 inhibitors, respectively;

FIGS. 12A and 12B show IC₅₀ values for erythromycin alone and in conjunction with VEGFR-1 inhibitors, respectively;

FIG. 13 shows the chemical structure of lisuride (S)(−);

FIG. 14 shows the chemical structure of miconazole;

FIG. 15 shows the chemical structure of sulconazole;

FIG. 16 shows the chemical structure of flutamide;

FIG. 17 shows the chemical structure of danazol;

FIG. 18 shows cell proliferation data for lisuride;

FIG. 19 shows cell proliferation data for miconazole;

FIG. 20 shows cell proliferation data for flutamide;

FIG. 21 shows cell proliferation data for danazol;

FIG. 22 quantifies measured HUVEC sprouting after treatment with select compounds;

FIG. 23 quantifies measured HUVEC sprouting after treatment with select compounds in conjunction with VEGF;

FIG. 24 quantifies measured HUVEC sprouting after treatment with select compounds and a VEGFR-1 inhibitor;

FIG. 25 shows animal weight data for in vivo experiments described herein;

FIG. 26 shows animal weight data for in vivo experiments described herein;

FIG. 27 shows animal weight data for in vivo experiments described herein;

FIG. 28 shows tumor growth data for in vivo experiments described herein;

FIG. 29 shows tumor growth data for in vivo experiments described herein;

FIG. 30 shows tumor growth data for in vivo experiments described herein;

FIG. 31 shows tumor data for in vivo experiments described herein;

FIG. 32 shows tumor data for in vivo experiments described herein;

FIG. 33 shows organ weight data for in vivo experiments described herein;

FIG. 34 shows organ weight data for in vivo experiments described herein;

FIG. 35 shows organ weight data for in vivo experiments described herein;

FIG. 36 shows vessel density data for in vivo experiments described herein;

FIGS. 37A through C show an excerpt from a data sheet corresponding to the field A1-AD36 which reproduces data from experiments described herein;

FIGS. 38A and B show an excerpt from a data sheet corresponding to the field A37-O72 which reproduces data from experiments described herein;

FIGS. 39A and B show an excerpt from a data sheet corresponding to the field A73-O95 which reproduces data from experiments described herein; and

FIG. 40 graphically represents selected fields of data from FIGS. 37-39.

### DETAILED DESCRIPTION OF THE INVENTION

The following examples serve only to illustrate the invention and are not intended to limit the same. If sources are not specifically described materials are known and commercially available.

It should be understood that the compounds of the present invention may have chemical and structural properties which are different from those specified hereinafter at some point during their manufacture and use. For example, during use, the compounds may undergo a series of metabolic changes that result in intermediaries with properties outside of the ranges set forth hereinafter for certain parameters. However, such intermediates and metabolic end products are nevertheless still within the scope of this invention if they at one time before, during, or after administration have the requisite values specified hereinafter.

As indicated above, compounds of the present invention can be used to affect angiogenesis. Such uses could be confined to cell or tissue culture application, as well as administration to a patient. In order to ensure the invention is fully disclosed to skilled workers, details related to the identification of the claimed compounds are provided by way of introduction herein. Where a description provides the purpose of a particular step or material, such purpose is based on the current understanding of the mechanism of action. Such description is provided in the interest of full disclosure; however, the invention is not bound to the theories described herein.

Primary Angiogenesis Screens

HUVEC Spheroid Evaluation

A test substance was selected for evaluation in angiogenesis screens. The test substance was synthesized or obtained as appropriate and stored at −20° C. in DMSO. Prior to use, the test substance was thawed and 5 mM of the substance was diluted to a 10 fold concentrated working solution (100 μM) in endothelial cell basal medium. The basal medium fraction was supplemented with 25 ng VEGF/100 μl for the stimulation of HUVEC sprouting.

The screening procedures were modified versions of those described by Korff and Augustin (J Cell Sci 112: 3249-58, 1999). By way of summary, spheroids were prepared by pipetting 400 human umbilical vein endothelial cells (HUVEC) into each well of nonadhesive 96-well plates. Prior to use the HUVEC were cultured according to the supplier's instructions (PromoCell, Heidelberg, Germany). The cells were allowed to aggregate overnight into spheroids (Korff and Augustin: J Cell Biol 143: 1341-52, 1998). After aggregation, 48 HUVEC spheriods were harvested. Each harvested spheroid was seeded in 900 μl of methocel-collagen solution and pipetted into an individual well of a 24-well plate to allow collagen gel polymerization.

Thirty (30) minutes after seeding, the test substance was added. The working dilution (10 fold concentration) was utilized, 100 μl was pipetted on top of the polymerized collagen gel in each well. The plates were then incubated at 37° C. for 24 hours. At the end of the incubation period the plates were fixed by adding 1 ml paraformaldehyde (10%) to each well.

The HUVEC spheriods were microscopically assayed for endothelial cell (EC) sprouting intensity. The degree of EC sprouting was classified into one of four groups by comparing the degree of sprouting to that observed in VEGF control cells. The groups were: strong inhibition of sprouting (indicating the test compound is a strong angiogenesis inhibitor), sprouting inhibited to basal sprouting (indicating that the test compound is an angiogenesis inhibitor), excessively above the VEGF control sprouting (indicating the test compound is an angiogenesis stimulator) or indistinguishable to the VEGF control sprouting (where the test compound appears to have no effect). For exemplary results, see FIG. 1.

NHDF Spheroid Evaluation

Where the test compound was classified as an inhibitor or stimulator, a second screening process was performed. In the second screen, the test substance was thawed and 5 mM of the substance was diluted to a 10 fold concentrated working solution (100 μM) in endothelial cell basal medium. No supplements were used in the basal medium.

Instead of HUVEC, spheroids were prepared by pipetting 400 human dermal fibroblasts (NHDF) into each well of nonadhesive 96-well plates. Prior to use the NHDF were cultured according to the supplier's instructions (PromoCell, Heidelberg, Germany). The cells were allowed to aggregate overnight into spheroids (Korff and Augustin: J Cell Biol 143: 1341-52, 1998). After aggregation, 48 NHDF spheroids were harvested. Each of the harvested spheroids was seeded in 900 μl of methocel-collagen solution and pipetted into an individual well of a 24-well plate to allow collagen gel polymerization.

Thirty (30) minutes after seeding, the test substance was added. The working dilution (10 fold concentration) was utilized, 100 μl was pipetted on top of the polymerized collagen gel in each well. The plates were then incubated at 37° C. for 24 hours. At the end of the incubation period the plates were fixed by adding 1 ml paraformaldehyde (10%) to each well.

The NHDF spheroids were microscopically assayed for fibroblast scattering intensity. The degree of fibroblast scattering intensity was classified into one of four groups: strong inhibition of scattering (indicating the test compound is a non-specific inhibitor), scattering inhibited or only mildly inhibited (indicating the test compound is an EC specific inhibitor), scattering unaffected (indicating the test compound is an EC specific stimulator) or scattering increased (indicating the test compound is a non-specific stimulator). For exemplary results, see FIG. 1.

Angiogenesis Inhibitors

Where a test compound reduced HUVEC sprouting to basal sprouting or there was no sprouting at all, and where NHDF scattering was not affected or mildly affected, the compound was deemed an inhibitor of angiogenesis.

Perhexiline Maleate

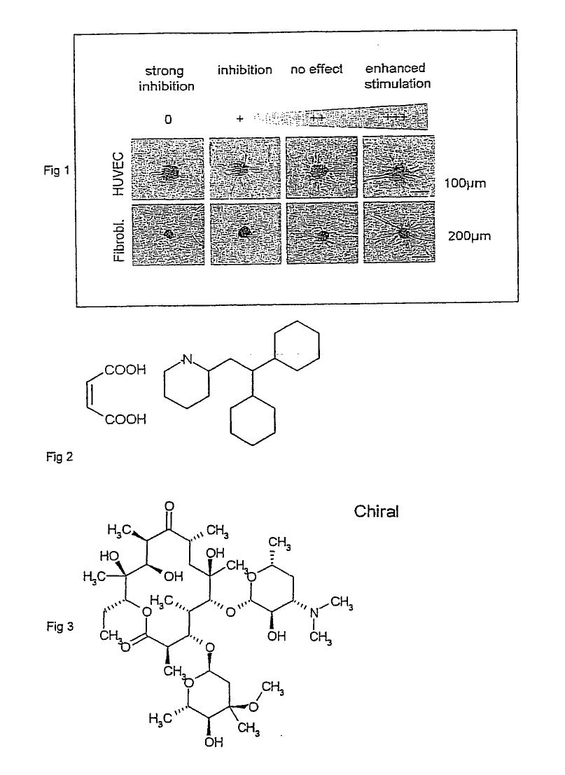

Perhexiline maleate is one such inhibitor. Its chemical structure is shown in FIG. 2. Its CAS No. is 6724-53-4, formulaic structure is C₂₃H₃₉NO₄, and molecular weight is 393.6. Perhexiline maleate exhibited sprouting inhibition but no fibroblast invasion effect, making it a specific angiogenesis inhibitor.

Erythromycin

Erythromycin is one such inhibitor. Its chemical structure is shown in FIG. 3. Its CAS No. is 114-07-8, formulaic structure is C₃₇H₆₇NO₁₃, and molecular weight is 733.9. Erythromycin exhibited sprouting inhibition but no fibroblast invasion effect, making it a specific angiogenesis inhibitor. This finding is surprising in light of the historical uses of erythromycin and even in light of more recent work with alternative applications of the compound.

By identifying the above-noted compounds as angiogenesis inhibitors, the present inventors make available new compounds that will inhibit or prevent angiogenesis. It may be desirable to prevent, decrease, or stop angiogenesis in a patient with certain disease states, such as cancer. Alternatively, it may be desirable to prevent angiogenesis in an otherwise healthy animal or cell/tissue culture model to further study the mechanisms of angiogenesis.

In Vitro Tests which Confirm Inhibition

Cell Proliferation Assay

In order to further confirm the surprising results described above, the compounds were tested in a cell proliferation assay. Crystal violet was used to measure cell proliferation; this basic dye stains cell nuclei and provides a sensitive, rapid evaluation. Light absorption of unstained or destained cell layers is negligible, making it possible to perform cell number measurements in wells.

Six cell types (HUVEC, HDMEC, SMC, Fibroblast, A375 and RENCA) were plated and treated with the compounds in a log₂ titration for 72 hours. The optimal cell number for each cell type was determined and optical density (OD) was measured at 595 nm. Each data point was measured in quadruplicate. The assay was stopped after 72 hours and subsequently analyzed.

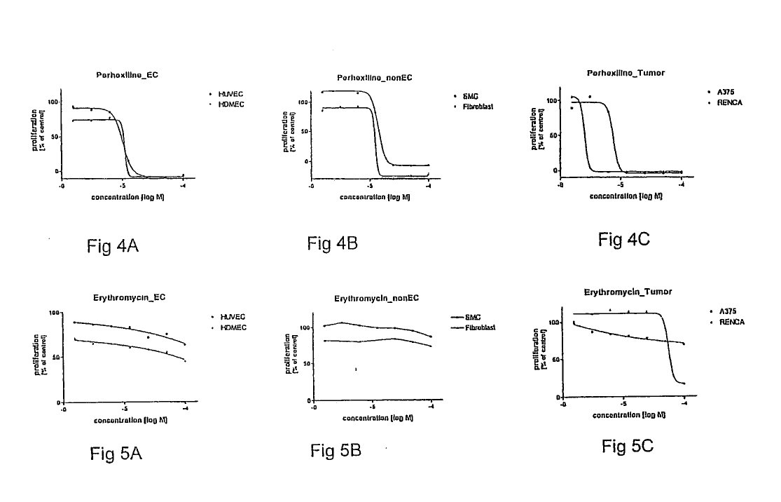

The OD-value of the plated cells before addition of the compounds was subtracted from all OD-values obtained after treatment. The percentage in relation to control proliferation was then calculated. Negative values therefore represent a count of fewer cells than were plated. The IC₅₀ is given below in Table 1. Results are shown as a percentage in relation to the control proliferation. The IC₅₀ were calculated using GraphPad Prism software (GraphPad Software, Inc.). Perhexiline results are depicted graphically in FIGS. 4A-4C and Erythromycin results in FIGS. 5A-5C.

TABLE 1
Summary of the IC₅₀ obtained in the proliferation assay.

| | IC₅₀ values for each cell type | | | | | |
|---|---|---|---|---|---|---|
| Compound | HUVEC | HDMEC | SMC | Fibroblast | A375 | RENCA |
| Perhexiline | 1.0 × 10⁻⁵ | 1.1 × 10⁻⁵ | 1.4 × 10⁻⁵ | 1.2 × 10⁻⁵ | 2.5 × 10⁻⁵ | 7.6 × 10⁻⁶ |
| Erythromycin | >1 × 10⁻⁴ | ˜1 × 10⁻⁴ | >1 × 10⁻⁴ | >1 × 10⁻⁴ | >1 × 10⁻⁴ | 5.6 × 10⁻⁵ |

Cytoviability Assay

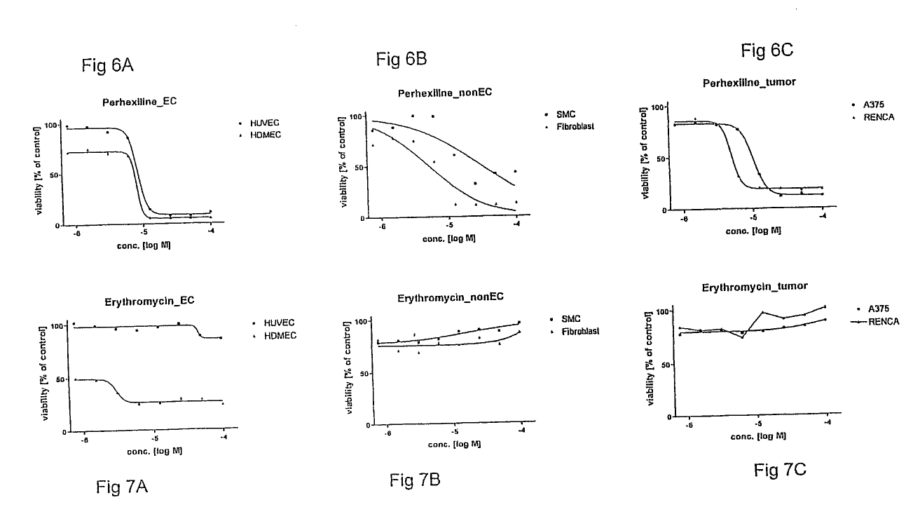

A cell viability assay based on cellular reduction of resazurin to the fluorescent product resorufin was performed. Viable cells can metabolize and reduce the dye, whereas dead cells rapidly lose the capacity to do so, making the increase in fluorescence directly proportional to the number of viable cells. The tested compounds were added in a log₂ titration and viable cells were analyzed 24 hours after treatment. The fluorescence was determined at an excitation wavelength of 560 nm and an emission wavelength of 590 nm. The percentage of viable cells in relation to the untreated control proliferation was calculated. Table 2 shows the IC₅₀ values obtained in the cell viability assay. Perhexiline results are depicted graphically in FIGS. 6A-6C and Erythromycin results in FIGS. 7A-7C.

TABLE 2
Summary of selected IC₅₀ results obtained in the cell viability assay

| | IC₅₀ values for each cell type | | | | | |
|---|---|---|---|---|---|---|
| Compound | HUVEC | HDMEC | SMC | Fibroblast | A375 | RENCA |
| Perhexiline | 8.4 × 10⁻⁶ | 8.0 × 10⁻⁶ | 3.2 × 10⁻⁵ | 5.2 × 10⁻⁶ | 1.0 × 10⁻⁵ | 5.0 × 10⁻⁶ |

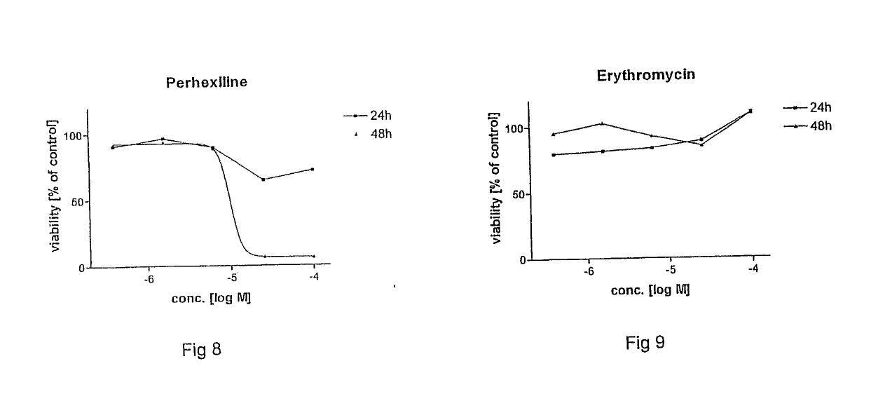

Through the two aforementioned assays it was determined that RENCA cells are more sensitive when treated with Perhexiline than the other cell types in the proliferation assay and in the viability assay. Therefore, the B16F10 cells were subsequently tested. The B16F10 tumor model, like the RENCA model, is an orthotopic tumor model. The IC₅₀ after 24 and 48 hours incubation for Perhexiline- and Erythromycin-treated B16F10 cells was determined at 1.0×10⁻⁵ M; results are given in FIGS. 8 and 9.

Evaluation of Synergistic Effects in Combination with VEGF Inhibitors

The 3D angiogenesis assay is suitable for IC₅₀ determinations with a narrow variation. Therefore, the assay was applied to test compounds alone or in combination with a VEGFR-2 inhibitor to address changes in IC₅₀. The inhibitors tested were SU5614 and PTK787/ZK222584. The VEGFR-2 inhibitor was applied at a concentration close to the IC₅₀.

SU5614 has an IC₅₀=4.2×10⁻⁶ M and has been described in, inter alia, Sun L, et al., Synthesis and biological evaluations of 3-substituted indolin-2-ones: a novel class of tyrosine kinase inhibitors that exhibit selectivity toward particular receptor tyrosine kinases. J Med Chem. Jul. 2, 1998;41(14):2588-603. and available from, e.g. Calbiochem. PTK787/ZK222584 (at times abbreviated AG1 in the figures) has an IC₅₀=1.0×10⁻⁷ M and has been described in, inter alia, Bold, G. et al., New anilinophthalazines as potent and orally well absorbed inhibitors of the VEGF receptor tyrosine kinases useful as antagonists or tumor-driven angiogenesis, J. Med. Chem. 2000, 43, 2310-2323.

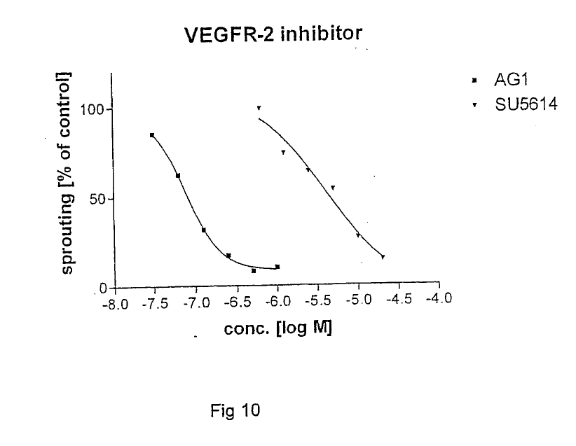

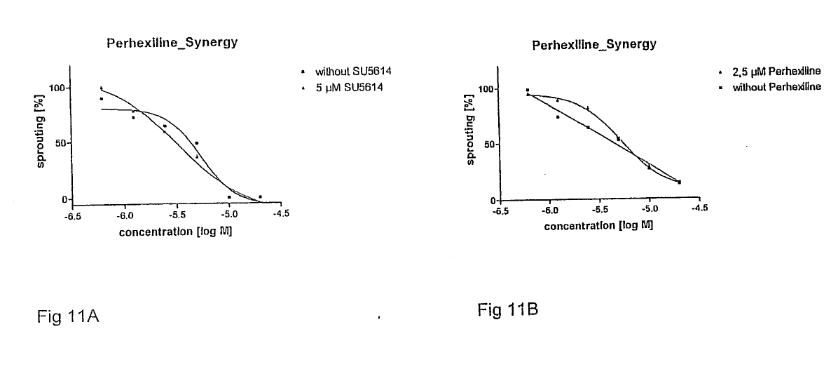

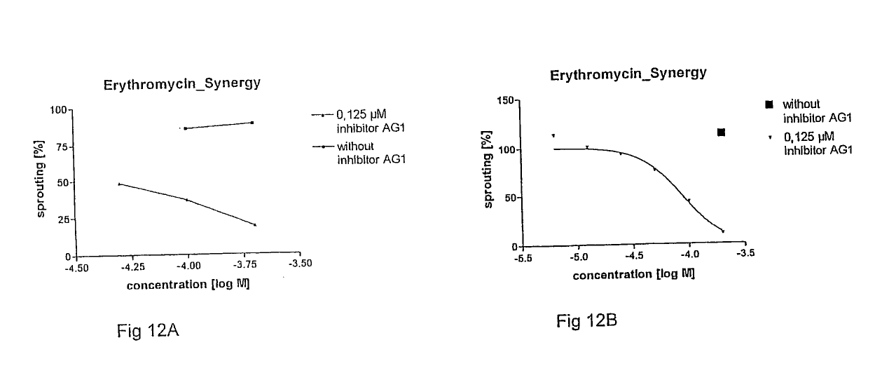

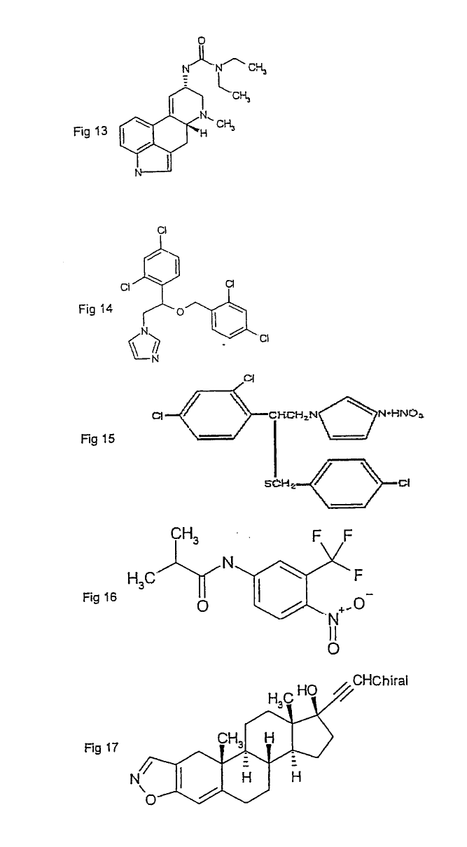

The IC₅₀ value of the VEGFR-2 inhibitors was determined in the spheroid-based 3D assay, see FIG. 10. The perhexiline or erythromycin was added on top of the gel where tested in combination with AG1 or directly in the gel when tested in combination with SU5614, at the concentration shown in FIGS. 11A and B and 12A and B, respectively. Data are summarized in Table 3.

TABLE 3
Summary of the IC₅₀ obtained in the
synergistic 3D angiogenesis assay

| | without VEGFR-2 | |
|---|---|---|
| Compound | inhibitor | with VEGFR-2 inhibitor |
| Perhexiline | 5.4 × 10⁻⁶ | 3.3 × 10⁻⁶ |
| Erythromycin | — | 8.6 × 10⁻⁵ |

Further assays can be performed to demonstrate the useful effects of the claimed compounds. For example, the Spherogenex angiogenesis assay can be used to elucidate the effects of specific enantiomers of the test compounds. As angiogenesis is known to be driven at times by VEGF, the assay can also be employed to ascertain bFGF-driven angiogenesis, thereby allowing determination of the effects of the compounds on non-VEGF-driven angiogenesis. mRNA expression profiling of the Spherogenex angiogenesis assay comparing treatment with the angiogenesis modulating compounds and nontreated controls could be used to determine which signalling pathways are affected by the test compounds.

In Vivo Confirmation of Surprising Effects

Another analysis which was performed is a Hepato-Cellular Carcinoma Model (HCC) in vivo study. The study evaluated the anti-angiogenic effect of Perhexiline in a subcutaneous and highly vascularized tumor model such as Alexander cells. Perhexiline had been shown, as described above, to affect in vitro 3D sprouting of HUVEC (IC₅₀=1.5×10⁻⁶ M) more potently than fibroblast scattering (IC₅₀=1.9×10⁻⁵ M). Perhexiline also has an influence on the viability of cells (Endothelial cells, Fibroblast, Alexander cells) with an IC₅₀ around 10 μM which could interfere with potential anti-angiogenic effects.

An example of how to repeat the analysis is to obtain Alexander cells (PLC/PRF/5) (available from, e.g. American Type Culture Collection, Manassas, Va., USA). Monolayers of Alexander cells can be grown in 85% Dulbecco's Modified Eagle's Medium (DMEM), 15% Fetal Bovine Serum (FBS), 4 mM L-glutamine, 100 units penicillinG/ml, and 100 μg of streptomycin-sulfate/ml. The cells can be cultured in a humidified atmosphere of 90% air and 10% carbon dioxide at 37° C. Media may preferably be changed every 4 days.

Six to eight week old female NMRI^{-nu/nu} mice could be utilized. The Alexander cells can be implanted in the left flank region of the mice, for example, by injecting 4×10⁷ cells in 0.2 ml aliquots into the subcutaneous space of the left flank using a 29 G needle syringe. After inoculation the appearing subcutaneous tumours can be measured by callipering and multiplying the distances of all three dimensions.

Therapy can be initiated after the tumor size has reached a volume of approximately 100 mm³ (day 1 of the study), and continue for about 21 days. Four experimental groups could be used as detailed in Table 4, below.

TABLE 4
Optional dosing scheme for in vivo analysis

| | | |
|---|---|---|
| Group | Treatment | Application Route |

| | | | |
|---|---|---|---|
| 1 | Control | — | — |
| 2 | Perhexiline | 40 mg/kg | p.o. |
| | | (1/2 MTD) | |
| 3 | Erythromycin | 10 mg/kg | i.p. |
| 4 | PTK787/ZK222584 | 50 mg/kg | p.o. |

As shown, Group 1 would serve as control, where the animals would receive no further treatment after injection of Alexander cells. Group 2 could evaluate perhexiline's effects on the tumor through daily doses of 40 mg/kg via oral gavage. Group 3 could receive erythromycin once daily in standard dose levels, such as 10 mg/kg i.p., preferably given in conjunction with a low dose of PTK787/ZK222584. Group 4 could be administered PTK787/ZK222584 orally at doses of 50 mg/kg twice daily, optionally a sub-group of Group 4 or a separate Group 5 could receive a low dose of PTK787/ZK222584.

After approximately 21 days of treatment, mice could be sacrificed and tumors collected and optionally stored appropriately at −80° C. For histological examination of the tumor vasculature, cryosections of the tissues (thickness of 5-10 μm) can be taken. For visualization of the blood vessels, immunohistochemical staining for CD 31 (PECAM-1) can be performed, and vessels counted microscopically using a defined magnification (×200). Examinations of all tissue sections at low magnification and can be carried out, including photography if desired. The proliferation index of the tumor tissue could be examined by BrdU labeling of cryosections. If that is the case, BrdU (500 mg/kg) should be administered to the animals 12 hours before sacrifice. To investigate the apoptotic index a TUNEL staining could be performed on cryosections.

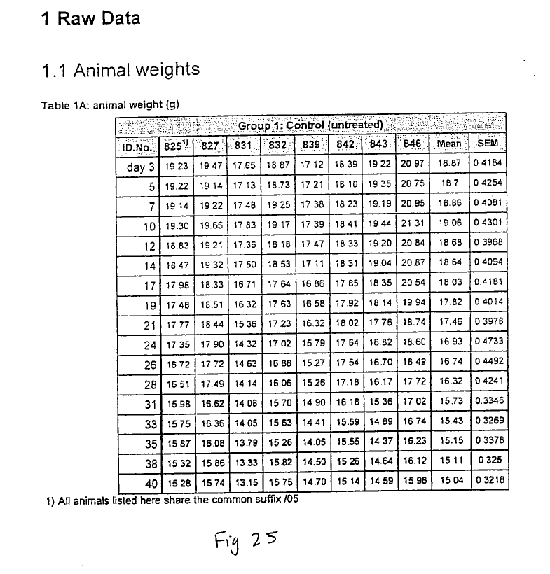

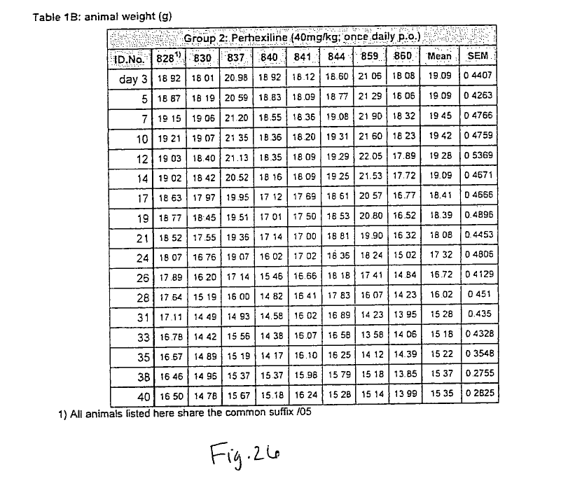

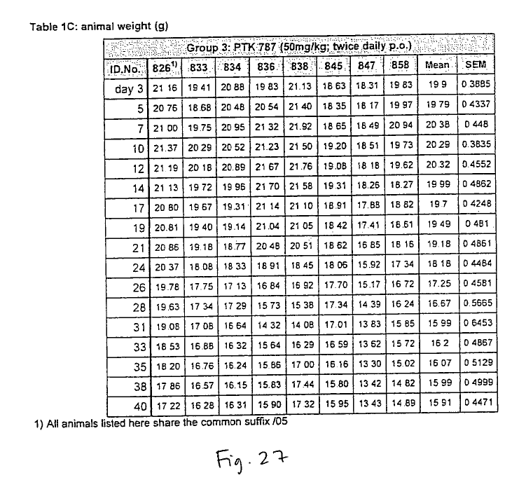

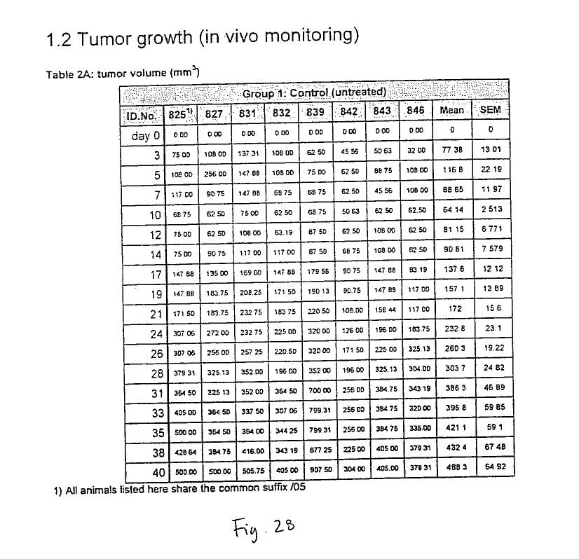

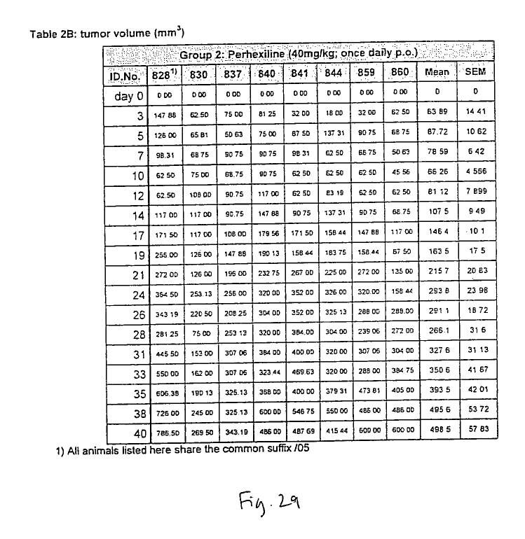

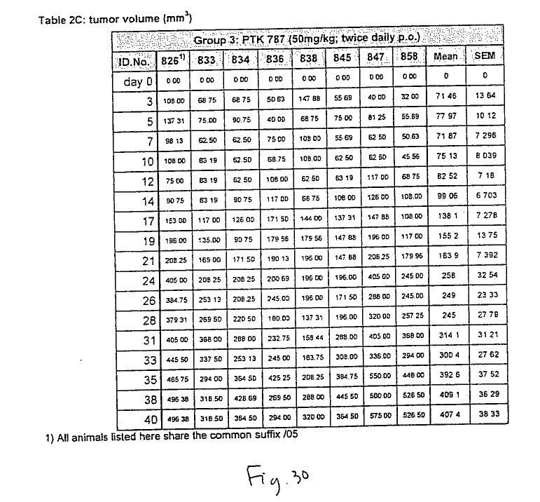

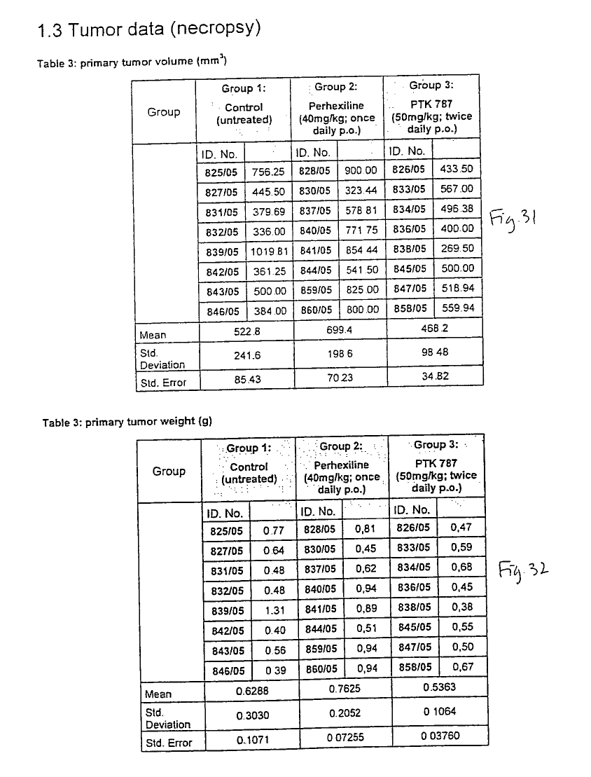

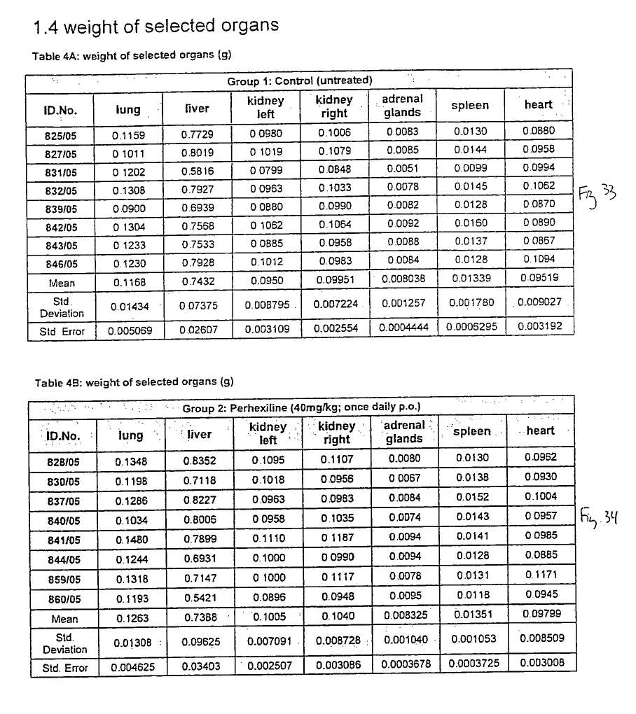

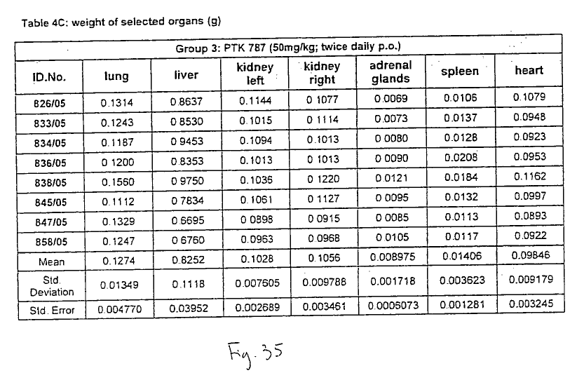

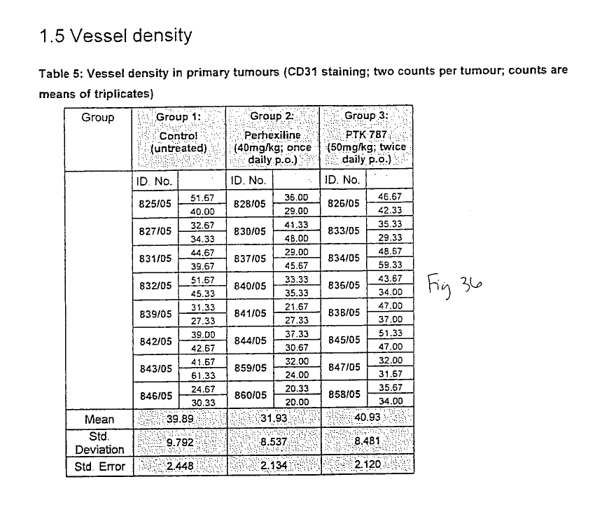

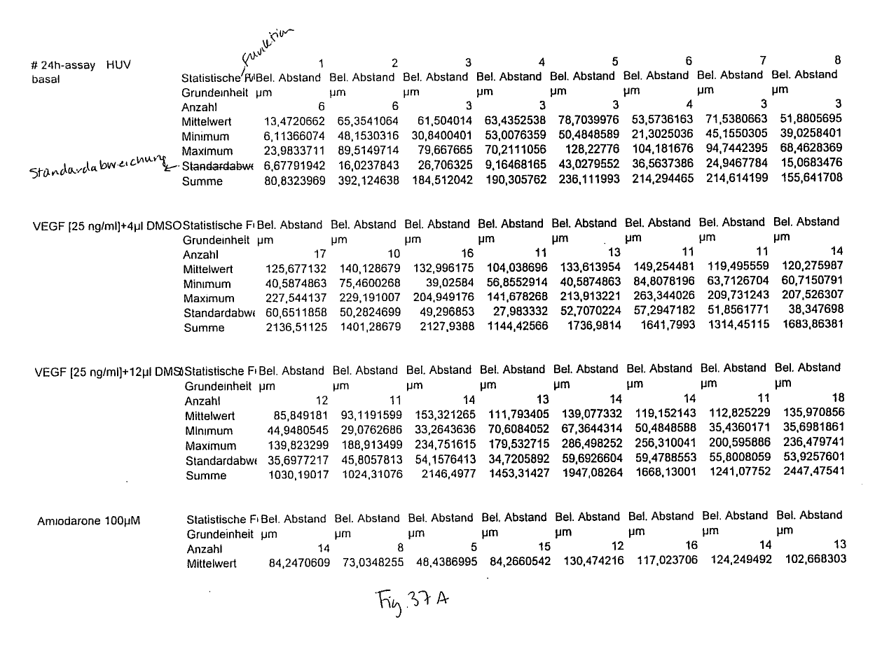

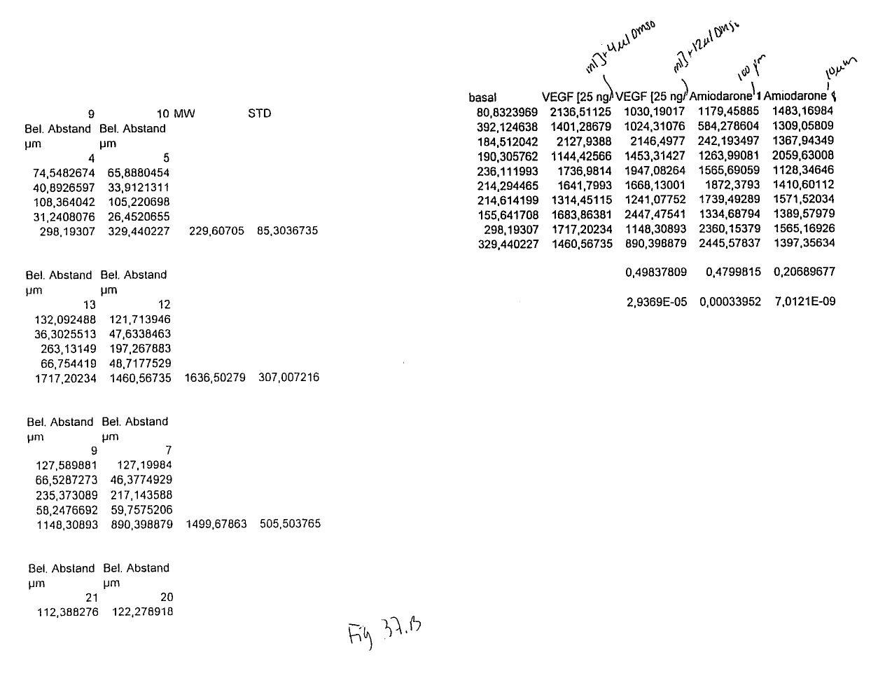

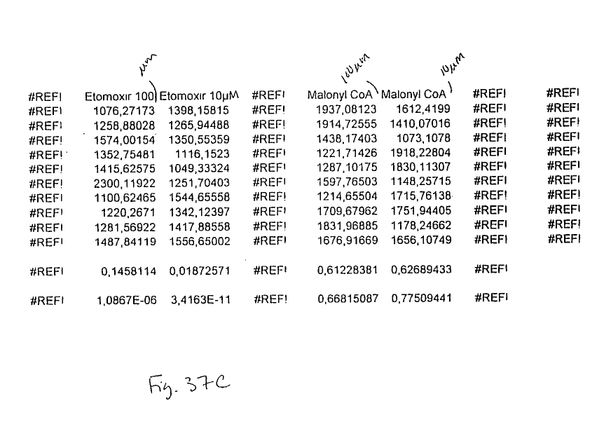

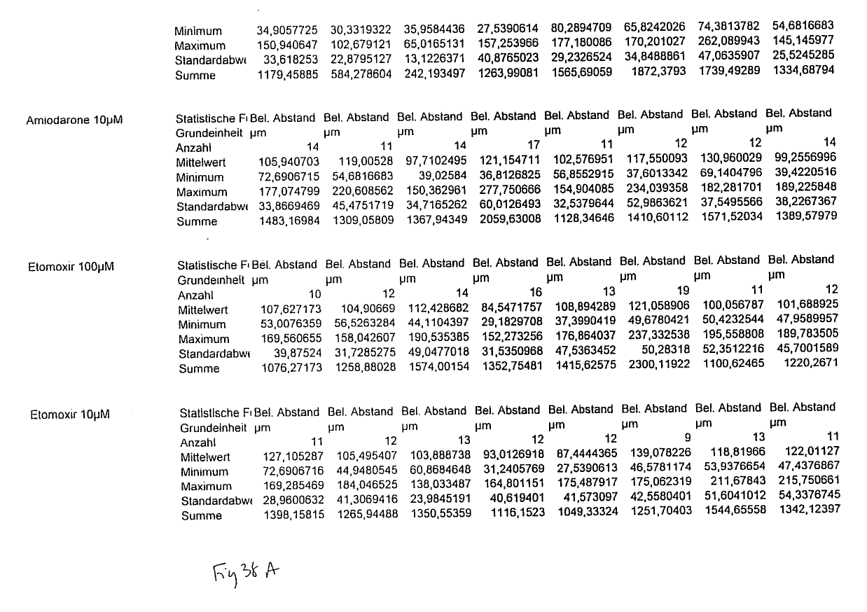

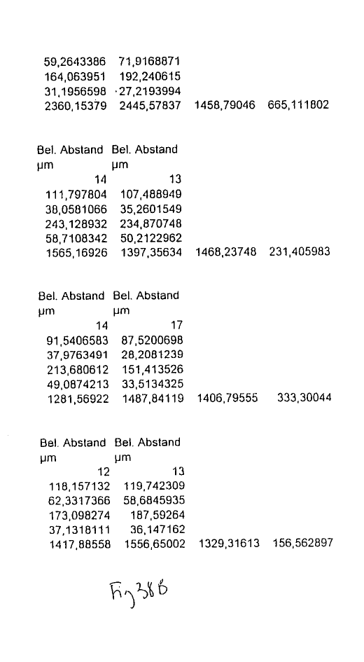

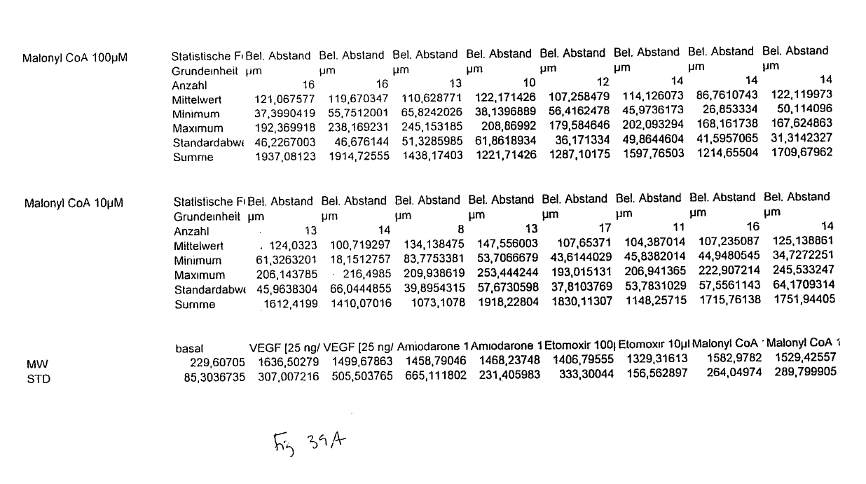

In the study that was performed, treatments were shown to have an effect on tumor size and vessel density; results are shown in FIGS. 25-36. As noted in particular in FIGS. 28-30, on average the tumor growth at many time points was reduced in the groups treated with Perhexiline or PTK 787 as compared to control. Also, as seen in FIG. 36, Perhexiline had a significant effect on vessel density. Thus, it has been shown that there is a surprising in vivo angiogenesis-inhibiting activity.

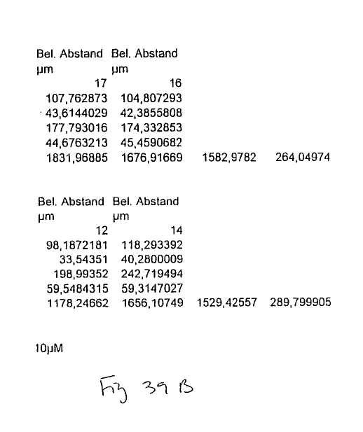

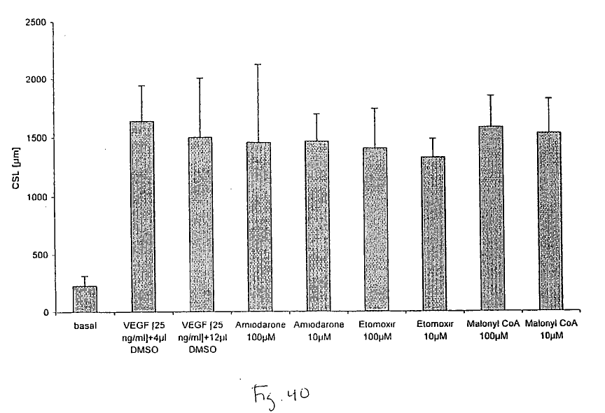

Further studies have evidenced that CPT-1 inhibitors other than Perhexiline fail to provide this effect. That is, the effects on tumor size and vessel density were not seen with Amiodarone Hydrochloride (Sigma-Aldrich: A8423, at 10 and 100 μm), Etomoxir (Sigma-Aldrich: E1905, at 10 and 100 μm). Malonyl-CoA (Sigma-Aldrich: 63410, at 10 and 100 μm), Trimetazidine dihydrochloride (Sigma-Aldrich: 653322), see FIGS. 37 A-40 (results for Trimetazidine dihydrochloride not shown). Perhexiline is unique in this respect.

Angiogenesis Stimulators

Where a test compound stimulated HUVEC sprouting excessively above the VEGF control and where NHDF scattering was unaffected or only mildly increased, the compound was deemed a stimulator of angiogenesis.

Dopamine Regulators

Bromocriptine is an angiogenesis stimulator with the molecular formula C₃₂H₄₀BrN₅O₅.CH₄SO₃ and a molecular weight of 750.70. Eticlopride is also known as a dopamine receptor blocker, as is Lisuride (S)(−). Lisuride's chemical structure is shown in FIG. 13. Its CAS No. is 18016-80-3, formulaic structure is C₂₀H₂₆N₄O, and molecular weight is 338.5. Lisuride (S)(−) exhibited increased sprouting stimulation but no fibroblast invasion effect, making it a specific angiogenesis stimulator. This is in surprising contrast to that described previously and noted above, where research indicated that certain of these dopamine regulators had no effect on angiogenesis or actually works to prevent angiogenesis.

Anti-Mycotics

Miconazole is one such stimulator. Its chemical structure is shown in FIG. 14. Its CAS No. is 22916-47-8, formulaic structure is C₁₈H₁₄Cl₄N₂O, and molecular weight is 416.1. Miconazole exhibited increased sprouting stimulation but no fibroblast invasion effect, making it a specific angiogenesis stimulator. Sulconazole, whose chemical structure is shown in FIG. 15, is (±)-1-[2.4-dichloro-b-[(p-chlorobenzyl)-thio]-phenethyl]imidazole mononitrate. Econazole is 1-[2-{(4-chloro-phenyl)methoxy}-2-(2,4-dichlorophenyl)ethyl]-1H-imidazole mononitrate, both are known anti-mycotics and have surprisingly been shown to be angiogenesis stimulators as well

Anti-Androgens

Flutamide is one such stimulator. Its chemical structure is shown in FIG. 16. Its CAS No. is 13311-84-7, formulaic structure is C₁₁H₁₁F₃N₂O₃, and molecular weight is 276.2. Flutamide exhibited increased sprouting stimulation but no fibroblast invasion effect, making it a specific angiogenesis stimulator. Danazol is also an anti-androgen that has surprisingly been shown to be an angiogenesis stimulator. Its chemical structure is shown in FIG. 17. Its CAS No. is 17230-88-5, formulaic structure is C₂₂H₂₇NO₂, and molecular weight is 337.5. Danazol exhibited increased sprouting stimulation but no fibroblast invasion effect, making it a specific angiogenesis stimulator.

By identifying the above-noted compounds as angiogenesis stimulators, the present inventors make possible the possibility of new compounds that will stimulate or begin angiogenesis. It may be desirable to stimulate angiogenesis in a patient with certain disease states, such as ischemia. Alternatively, it may be desirable to stimulate angiogenesis in an animal or cell/tissue culture model to further study the mechanisms of angiogenesis. For example, by administering a compound of the present invention, angiogenesis could be stimulated in an animal model expressing a tumor, providing additional research opportunities into the growth of the tumor and possibly improved animal models for studying tumor treatments.

In Vitro Tests which Confirm Stimulation

Cell Proliferation Assay

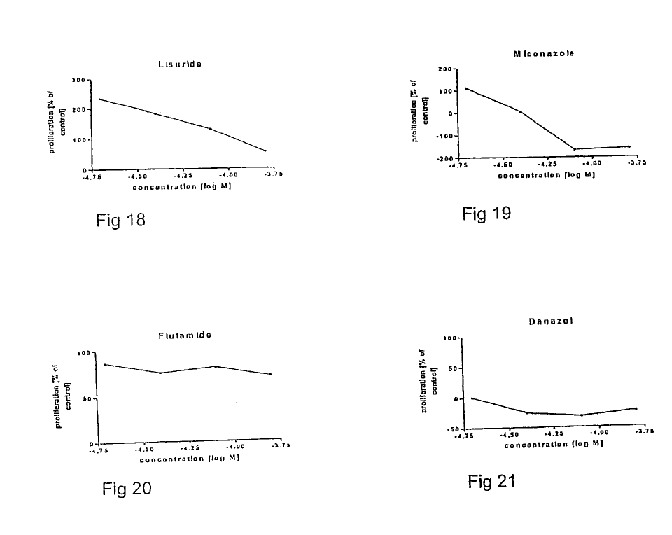

In order to further confirm the surprising results described above, the compounds were tested in a cell proliferation assay. The assay was performed as described, above. The OD value at day 0 was subtracted from the OD value after 72 hours of treatment. Negative values therefore represent observation of fewer cells than originally plated and further indicate cytotoxic effects of the compound on HUVEC. The compounds inhibit proliferation, results are shown in FIGS. 18-21.

HUVEC Spheroid Results

As described above with regard to angiogenesis inhibitors, the compounds newly found to be angiogenesis stimulators were also evaluated in a 3D angiogenesis assay. They were all consistently found to independently stimulate EC sprouting or further increase VEGF-induced sprouting.

The HUVEC monolayer was treated for 6 hours with the compounds then the induced transcriptional changes, when compared to untreated HUVEC, were determined. The compounds were tested according to their ability to induce sprouting without addition of VEGF, in combination with VEGF and together with the VEGFR-2 inhibitor PTK787/ZK222584 (1 μM). The VEGFR-2 inhibitor completely prevents VEGF-induced sprouting at a concentration of 1 μM.

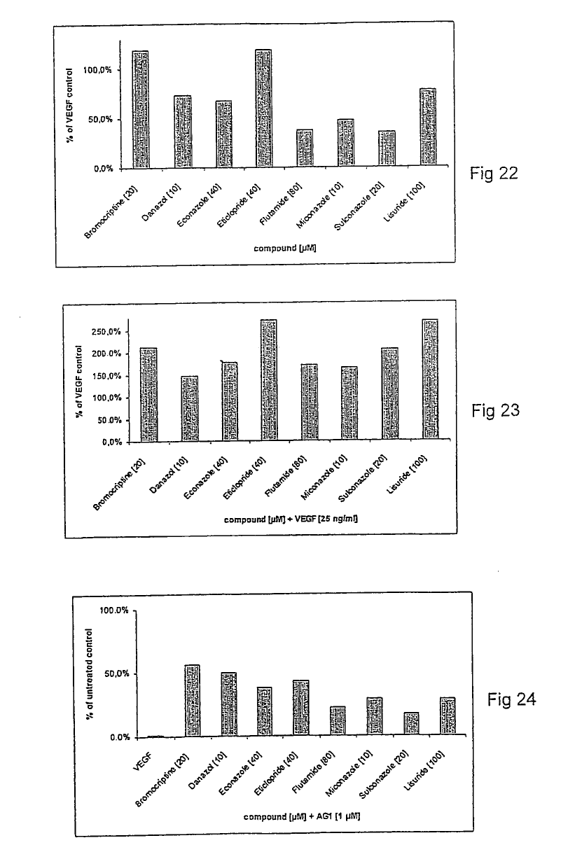

FIG. 22 shows the effects of the compounds alone on HUVEC sprouting, after 24 hours of treatment the basal sprouting was subtracted and the resultant sprouting compared to VEGF-induced sprouting. Both bromocriptine and eticlopride stimulated sprouting in excess of the VEGF-induced sprouting.

The compounds given in combination with VEGF (25 ng/ml) were also evaluated, data shown in FIG. 23. The compounds can enhance VEGF-driven angiogenesis even further. Results from the evaluation of the compounds administered in conjunction with PTK787/ZK222584 are shown in FIG. 24, where the percentage is given as compared to untreated control. The results show that the sprouting is not VEGF-dependent since it can not be inhibited by VEGFR inhibitor.

In Vivo Tests to Further Elucidate Stimulation

In addition to the in vitro procedures noted above, in vivo tests could also be conducted to further qualify and quantify the action of the test compounds. The test compounds could be evaluated in conjunction with or without angiogenic stimulators (for example, growth factors such as VEGF or bFGF) in a matrigel angiogenesis assay as described in Passaniti, A., et al Lab Invest. 1992 October;67(4):519-28. Alternatively or in addition, the modified matrigel angiogenesis procedure as in Guedez, L., et al., American Journal of Pathology, 2003, vol. 162, no. 5, 1431-39 could be used.

Information could also be compiled by analysing the effects of test compound treatment on the angiogenesis process in disease models of myocardial infarction and tissue ischemias. Such models are known to the skilled worker and include, for example, those described in WO03/000009 (PCT/US02/19568) and Witzenbichler B., Am J Pathol. 1998 August;153(2):381-94.

A further in vitro evaluation that could be performed would be to evaluate the retinal vascularization and vessel density in mice treated with the compounds as described in WO 05/008250.

Dosing and Guidelines for Use

The compounds described herein can be used to affect angiogenesis. The amount of compound, route of administration and other related factors could well be based on the regimen advised for the compound's original indication. Such information is available to the skilled worker; see, for example, Goodman & Gilman, The Pharamacological Basis of Therapeutics. Dosing and delivery could be modified based on the needs of the patient and the availability of improved dosing techniques as they develop in the art.

The preferred method of treatment may further include a step of detecting angiogenesis in the affected tissue or organism following treatment. Methods of detecting angiogenesis are known in the art, see for example, U.S. Pat. No. 6,689,807.

It is contemplated that a compound of the present invention could be metabolised by enzymes in the body of the organism such as a human being to generate a metabolite that can affect angiogenesis. Such metabolites are within the scope of the present invention. It is also contemplated that precursor compounds could be administered which, after undergoing processing such as enzyme metabolism, would result in the compound of the present invention.

In certain circumstances, a skilled worker may choose to employ one or more of the compounds discussed above in conjunction with another active compound. Alternatively, two or more of the compounds disclosed herein may be administered in conjunction to affect angiogenesis to a different degree. The compounds may be administered at the same time or in a sequential fashion. One way to determine whether particular combinations of compounds interact in a preferred fashion is by preparing a linear isobologram (Tallarida R J., et al, Statistical analysis of drug-drug and site-site interactions with isobolograms, Life Sciences. 45(11):947-61, 1989). Such a diagram helps elucidate where the additional compound(s) have merely an additive effect, or where they have a synergistic effect. In such an evaluation, at least nine experimental groups are evaluated; three groups define the dose-response of a first compound, three groups define the dose-response for a second compound, and three groups define the dose-response for a fixed ratio combination of those two compounds.

The selected dose response values, ED₅₀ values, for the single dose groups are plotted as a line which assumes an addititive effect. This line is based on the compound ratio tested; a 1:2 ratio of first test compound to second test compound is shown. When data measured for the group with combined compound administration is superimposed on the hypothetical addititive line, an observed dose response which lies to the left (i.e., below) of the additive line represents a synergistic effect of the compounds used together, whereas a observed dose response to the right (i.e., above) of the additive line represents an antagonistic effect of the compounds in combination.

The compounds disclosed herein may be particularly effective when combined with treatments which block VEGF activity or signalling. Such anti-VEGF treatments include inhibitory anti-VEGF receptor antibodies, soluble receptor constructs, antisense strategies, RNA aptamers against VEGF and low molecular weight VEGF receptor tyrosine kinase (RTK) inhibitors.

Examples of specific compounds which may preferably be used in combination with the above-noted compounds are AVASTIN (bevacizumab), a recombinant human antibody to VEGF, and MACUGEN (pegaptanib sodium), an aptamer which selectively binds to and neutralizes VEGF. Research in the anti-VEGF art is ongoing, future compounds would be expected to show similar positive effects when used in conjunction with one or more of the compounds disclosed hererin.

The effective dosage amount will vary with the particular condition being treated, the age and physical condition of the subject being treated, the severity of the condition, the duration of treatment, the nature of concurrent therapy (if any), the specific route of administration and factors within the knowledge and expertise of a health care practitioner. For example, in connection with occlusive or obstructive vascular disorders, an effective amount is that amount which engenders sufficient angiogenesis so as to provide an increase in blood flow in the ischemic region.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method of inhibiting angiogenesis in a group of cells, a tissue or an organism, comprising administering to the group of cells, tissue or organism a therapeutically effective amount of Perhexiline, or pharmaceutically acceptable prodrugs, salts, solvates, hydrates, active metabolites, or combinations thereof.

2. A method according to claim 1, wherein the organism is a mammal.

3. A method according to claim 2, wherein the mammal has a condition selected from the group consisting of: cancer, sarcomas, retinopathy, macular degeneration, corneal ulceration, scleroderma, Berger's disease, proliferative vitreoretinopathy, chronic inflammation, inflammatory bowel disease, psoriasis, sarcoidosis, and rheumatoid arthritis.

4. A method according to claim 1, wherein the compound is administered locally.

5. A method of inhibiting angiogenesis in a group of cells, a tissue or an organism, comprising administering a therapeutically effective amount of a pharmaceutical composition comprising Perhexiline or pharmaceutically acceptable prodrugs, salts, solvates, hydrates, active metabolites, or combinations thereof to the group of cells, tissue or organism.

6. A method according to claim 5, wherein the organism is a mammal.

7. A method according to claim 6, wherein the mammal has a condition selected from the group consisting of: cancer, sarcomas, retinopathy, macular degeneration, corneal ulceration, scleroderma, Berger's disease, proliferative vitreoretinopathy, chronic inflammation, inflammatory bowel disease, psoriasis, sarcoidosis, and rheumatoid arthritis

8. A method according to claim 5, wherein the compound is administered locally.

9. A method according to claim 5, wherein the pharmaceutical composition is formulated as a salve, gel, ointment, patch, injection, oral solution or suspension.

10. A method according to claim 5, wherein the pharmaceutical composition is in a controlled release matrix.

11. A method according to claim 5, wherein the pharmaceutical composition further comprises a pharmaceutically acceptable carrier, diluent, vehicle, or excipient.

12. A method according to claim 5, further comprising administering at least one anti-VEGF treatment to the group of cells, tissue, or organism.

13. A method according to claim 1, wherein said at least one anti-VEGF treatment is selected from the group consisting of AVASTIN and MACUGEN.

14. A method of stimulating angiogenesis in a group of cells, a tissue or an organism, comprising administering a therapeutically effective amount of a compound selected from the group consisting of: Bromocriptine, Eticlopride, Lisuride (S)(−), Miconazole, Sulconazole, Econazole, Flutamide and Danazol or pharmaceutically acceptable prodrugs, salts, solvates, hydrates, active metabolites, or combinations thereof to the group of cells, tissue or organism.

15. A method according to claim 14, wherein the organism is a mammal.

16. A method according to claim 14, wherein the mammal has a condition selected from the group consisting of: stroke, ischemic heart disease, wound healing, ischemia, myocardial infarction, myocardosis, angina pectoris, unstable angina, coronary arteriosclerosis, arteriosclerosis obliterans, and cerebral infarction.

17. A method according to claim 14, wherein the compound is administered locally.

18. A method of stimulating angiogenesis in a group of cells, a tissue or an organism, comprising administering a therapeutically effective amount of a pharmaceutical composition comprising a compound selected from the group consisting of: Bromocriptine, Eticlopride, Lisuride (S)(−), Miconazole, Sulconazole, Econazole, Flutamide and Danazol or pharmaceutically acceptable prodrugs, salts, solvates, hydrates, active metabolites, or combinations thereof to the group of cells, tissue or organism.

19. A method according to claim 18, wherein the organism is a mammal.

20. A method according to claim 18, wherein the mammal has a condition selected from the group consisting of: stroke, ischemic heart disease, wound healing, ischemia, myocardial infarction, myocardosis, angina pectoris, unstable angina, coronary arteriosclerosis, arteriosclerosis obliterans, and cerebral infarction.

21. A method of increasing blood flow to a tissue, comprising administering a pharmaceutical composition comprising a compound selected from the group consisting of: Bromocriptine, Eticlopride, Lisuride (S)(−), Miconazole, Sulconazole, Econazole, Flutamide and Danazol to the tissue in an amount effective to promote angiogenesis.

22. A method according to claim 5, wherein said at least one anti-VEGF treatment is selected from the group consisting of AVASTIN and MACUGEN.
